(19) United States
(12) Patent Application Publication
(10) Pub. No.: US 2010/0189827 A1
(43) Pub. Date: Jul. 29, 2010

(54) ANTIVIRAL COMPOSITION COMPRISING ALNUS JAPONICA EXTRACTS

(75) Inventors: Hyuk Joon Kwon, Seoul (KR); Sun Hee Cho, Gyeonggi-do (KR); Sun Joong Kim, Gyeonggi-do (KR); Young Jin Ahn, Gyeonggi-do (KR); Jeong Chan Ra, Gyeonggi-do (KR)

Correspondence Address: INTELLECTUAL PROPERTY / TECHNOLOGY LAW, PO BOX 14329, RESEARCH TRIANGLE PARK, NC, 27709, US

(73) Assignee: RNL BIO CO., LTD., Seoul (KR)

(21) Appl. No.: 12/754,443

(22) Filed: Apr. 5, 2010

Related U.S. Application Data
(62) Division of application No. 11/570,678, filed on Dec. 29, 2006.

(30) Foreign Application Priority Data
Jun. 29, 2006 (KR) 10-2006-0059188

(51) Int. Cl. A61K 36/00; A61P 31/16

(52) U.S. Cl. 424/769

(57) ABSTRACT

Antiviral compositions including Alnus japonica extracts are described as useful for preventing or treating diseases caused by influenza virus infection of humans and other mammalian and avian subjects (e.g., pigs, horses, birds, and the like). Alnus japonica extracts in such use exhibit low toxicity in normal cell environments, and excellent antiviral effects. Compositions and anti-viral agents for influenza virus that include Alnus japonica extracts are effectively used in foods and pharmaceutical products for preventing and treating influenza virus diseases.

### CROSS-REFERENCE TO RELATED APPLICATIONS

This application is a divisional of U.S. patent application Ser. No. 11/570,678, filed on Dec. 14, 2006, which, in turn, is a U.S. national phase application, filed under the provisions of 35 U.S.C. §371 and claims the priority of International Patent Application No. PCT/KR2006/003847 filed Sep. 27, 2006 and published as International Publication No. WO 2008/001976 on Jan. 3, 2008, which claims the priority of Korean Patent Application No. 10-2006-0059188 filed Jun. 29, 2006. The disclosures of said International Patent Application and Korean Patent Application are herein incorporated by reference, in their respective entireties, for all purposes.

### TECHNICAL FIELD

The present invention relates to an antiviral composition comprising Alnus japonica extracts, more specifically, relates to a composition for preventing or treating diseases caused by influenza virus which infects human, pig, horse, bird, and the like.

### BACKGROUND ART

Virus cause various diseases, particularly, a typical one among pathogenic viruses that become a problem in the field of stockbreeding is Avian influenza virus. Avian influenza virus belongs to the orthomixoviridae family, and cause damage to poultry such as chicken, turkey. Avian influenza viruses are classified into 3 types of high-pathogenic, low-pathogenic and non-pathogenic Avian influenza viruses according to the degree of pathogenicity, among which the high-pathogenic virus is classified into “grad A” in the World Organization for Animal Health (OIE) and “the first level domestic animal infectious disease” in Republic of Korea.

The influenza virus is classified into type A, B or C according to the antigenicity of nucleocapsid protein and matrix protein. Moreover, according to the difference of antigen structure of haemagglutinin (HA) and neuraminidase (NA), the HA is classified into 16 subtypes and NA is classified 9 subtypes, wherein HA helps host cell receptor binding, and fusion between host cell membrane and viral envelope to cause a virus infection and NA plays an important role when virus buds out through the cell membrane after proliferation. Theoretically, 144 kinds of virus subtypes could exist by the combination of two proteins. Infection generally occurs by contact with bird secretions, furthermore, it is spread through the air, in both particle and droplet forms, human feet, vehicle delivering feedstuff, apparatus and feces on the surface of eggs etc.

Although there are various symptoms according to the pathogenicity of infecting virus, generally, they are respiratory symptoms, diarrhea and a sharp decline in egg production ratio etc. Moreover, in some cases, cyanosis appears at the head region such as crest, edema appears on the face, or feathers are ruffled. Mortality rate also varies from 0% to 100% according to pathogenicity, but since the symptoms are similar to those of Newcastle Disease, infectious larynogotracheitis, mycoplasma infection and the like, an accurate diagnosis is required.

High-pathogenic avian influenza had occurred 23 times from 1959 to 2003 throughout the world, most of them were endemic and contained. Outbreaks of H5N1 subtype high-pathogenic avian influenza had occurred in Korea in December 2003 occurred in more than 30 countries including Europe, Africa and most countries in Southeast Asia such as Japan, China, Thailand, Vietnam and Indonesia, thus becoming pandemic. Though it is known that human cannot get infected with avian influenza, prevention of Avian influenza is being of paramount importance to public health sector due to the case of human infection with H5N1 in 1997, isolation of H9N2 Avian Influenza viruses from humans in 1999 in Hongkong and human cases of H7 avian influenza infection in 2004 in Canada. According to a report of the World Health Organization (WHO), (http://www.who.int/csr/disease/avian_influenza/country/cases_table_{—}2006_{—}06_{—}20/en/index.html), it was confirmed that the 228 persons were infected with H5N1 subtype and 130 persons of them died during the period of 2003 to Jun. 20, 2006 in 10 countries. In Korea, since low-pathogenic Avian Influenza by H9N2 subtype had occurred in 1996, it reoccurred in 1999 and now it has been occurring throughout the whole country.

If avian influenza occurs, in most countries, poultry were slaughtered, and countries which have confirmed outbreaks of avian influenza cannot export poultry products to cause swinging damages into poultry industry. Furthermore, when there is a risk of human infection, the damages spread to the whole industry including the tourist industry and the transport industry, thus causing astronomical loss including.

Natural substances mean, which is not added with artificial factors, and the natural substances classified as GRAS (Generally Recognized As Safe) can be used without restrictions on the quantity thereof or foods in which natural substances are to be used. In domestic industry, the natural substances are classified as natural additives, to be used as food additives, and in foreign countries, it has been used as health foods and medical supplies without extra limitation for user's purpose, because of its excellent functionality.

Meanwhile, Alnus japonica is a dicotyledon and a deciduous tree is classified in the division, order Fagales, family Betulaceae, which is commonly called Alnus japonica tree. It grows near swamp, its height is about 20 m and its bark is of a deep purplish-brown color. Its winter bud is a long oval shape just like the shape of an egg turned upside down, which has three ridge lines and a peduncle. The leaves of Alnus japonica grow alternately, and they are oval shaped, egg-shaped (more or less rounded at both ends, widest at the bottom) or lanceolate. Both sides of a leaf are lustrous and leaf margins are saw-toothed.

The flower of Alnus japonica blooms in March˜April, is unisexual and forms a catkin. Staminate spike bears staminate flower and each bract has three˜four flowers. There are four perianths and four stamens in each flower. Fruit ripens in October and 2˜6 fruits are produced. It is long egg-shaped and looks like a pine cone. They are distributed in Korea, Japan and China, etc.

Recently, many researchers endeavor to develop anti-viral agents throughout the world. Lamibudine used for the treatment of HIV (Human Immunodeficiency Virus)-1 and hepatitis B, gancyclovir used for the treatment of symptoms of herpes virus infection, ribavirin which is used mainly for the treatment of symptoms of respiratory syncytial virus infection but can be used for the treatment of symptoms of various virus infection when it is an emergency and zanamivir Relenza® and oseltamivir, TAMIFLU™ which are synthesized artificially as neuraminidase inhibitors of influenza virus are all commercially available after getting approval. However, use of amantadine and its analogue, rimantadine, which are approved for treatment of influenza virus A has decreased for the appearance of resistant virus and its side effect. Recently, virus resistant to oseltamivir among H5N1 avian influenza viruses appeared, therefore, developments of various anti-virus agents are urgently required.

Therefore, the present inventors have made an extensive effort to develop a natural substance having a low toxicity to a normal cell, while having an excellent effect to inhibit proliferation of influenza virus. As a result, they found that a composition comprising Alnus japonica extracts have an anti-influenza virus effect, thereby completing the present invention.

### SUMMARY OF THE INVENTION

The present invention, in one aspect, relates to a food composition for preventing influenza virus diseases, comprising Alnus japonica extracts.

The present invention, in another aspect, relates to a pharmaceutical composition for preventing or treating influenza virus diseases, comprising Alnus japonica extracts.

The present invention, in another aspect, relates to an anti-viral agent for influenza virus, comprising Alnus japonica extracts as an active ingredient.

Other features and examples of the present invention will be clarified from the minute description and the appended claims as follows.

### DETAILED DESCRIPTION OF THE INVENTION, AND PREFERRED EMBODIMENTS

In the present invention, after a composition containing Alnus japonica extracts was added to SPF embryonated egg infected with Avian influenza virus and cultured, the plate hemagglutination test was performed, and as a result, it was confirmed that the composition containing Alnus japonica extracts has excellent anti-viral effect.

In one aspect, the present invention relates to a food composition for preventing diseases caused by influenza virus belonging to the orthomixoviridae family, comprising the Alnus japonica extracts and a sitologically acceptable supplemental additive.

In another aspect, the present invention relates to a pharmaceutical composition for preventing or treating diseases caused by influenza virus belonging to the orthomixoviridae family, comprising Alnus japonica extracts as an active ingredient.

In another aspect, the present invention relates to an anti-viral agent for influenza virus, comprising Alnus japonica extracts as an active ingredient.

In the present invention, said influenza virus is preferably selected from the group consisting of: human influenza virus, Swine influenza virus, Equine influenza virus, and Avian influenza virus. More preferably, said Avian influenza virus is KBNP-0028 (KCTC 10866BP).

### EXAMPLES

Hereinafter, the present invention will be described in more detail by examples. However, it is obvious to a person skilled in the art that these examples are for illustrative purpose only and are not construed to limit the scope of the present invention.

### Example 1

### Preparation of Alnus Japonica Extracts

The leaves, stems (duramen and bark of a tree), and flowers of Alnus japonica were picked, dried at room temperature for 24 hrs, chopped up and crushed. The obtained powder was added with 99.9% methanol, stirred for 24 hrs at room temperature to extract and vacuum-filtered to collect supernatant liquid, followed by eluting useful components from the obtained powder. The useful components are dried for 24 hrs at room temperature, and dissolved in 99.9% dimethyl sulfoxide (DMSO) solution to 20 mg/ml, thus preparing Alnus japonica extracts.

Although the Alnus japonica extracts according to the present invention could be obtained by the above described method, those distributed from The Korea Plant Extract Bank were used.

### Example 2

### Examination of Anti-Viral Effect of Alnus Japonica Extracts

### 2-1: Preparation of KBNP-0028

As avian influenza virus used in the experiment, hyperproliferative KBNP-0028 (KR 2006-0026591) cloned after subculturing A/chicken/Korea/SNU0028/2000(H9N2) virus (it is isolated in Korea in 2000) in chick embryo was used. That is, SNU0028 [A/chicken/Korea/SNU0028/2000(H9N2); isolation and report to National Veterinary Research and Quarantine Service, May 9, 2005] is low-pathogenic Avian Influenza virus of H9N2 subtype, isolated from chicken showing mortality and egg drop syndrome. The virus was isolated in a chicken farm located in North jeola Province in Jan. 28, 2000.

The isolation method is as follows: after kidney and tracheal sample from infected chicken are dissolved, suspended in phosphate buffer, and filtrated with 0.45 μm diameter filter paper, each sample is inoculated into three allantoic cavities of SPF (Specific Pathogen Free) embryonated egg (Sunrise Co., NY), and cultured at 37° C. to obtain allantoic fluid. The 20 μl of allantoic fluid and 20 μl of 0.1% chicken red blood cells, extracted from a chicken obtained after hatching the SPF embryonated egg, are dropped on glass plate, and mixed to carry out the plate hemagglutination test.

As a result, all of the allantoic fluids, obtained by inoculating kidney sample and tracheal sample, formed the hemagglutination. The virus was identified with RT-PCR and the analysis of base sequence using H9N2 specific primer (Kim Min Chul, Master's Thesis, 2002, Seoul National University), and stored at −70° C. Among them, the virus isolated from tracheal sample was used in the experiment.

In order to select a vaccinia strain having high productivity of embryonated egg, the SNU0028 was diluted with phosphate buffer to the concentration of 0.05 to 0.5 HAU/ml. 200 μl of the diluted solution was inoculated into 10-11-day-old SPF hatchery egg (Sunrise Co., NY) via the allantoic cavity, and the egg was cultured for three days at 37° C. Everyday, the embryonated eggs, which died three days ago, was discarded through egg examination in the morning and afternoon. The embryonated eggs, which survived for three days, were stored for 12-24 hrs at 4° C., from which allantoic fluid was collected to measure each of volume and hemagglutination titer thereof. Among them, allantoic fluid having the most quantity and the highest hemagglutination titer was inoculated into embryonated eggs using the same method as described above, and the eggs were subcultured 19 times to eggs whose productivity was increased due to high hemagglutination titer and high yield of allantoic fluid and thus they are named KBNP-0028. It was deposited at GenBank located Eoeundong, Youseonggu, Daejeon city, Korea on Oct. 26, 2005 (KCTC 10866BP).

### 2-2: Culturing Hatchery Egg Shell Fragments

The egg shell of 10˜11 day-old SPF hatchery egg (Sunrise Co., NY) was washed with 70% ethanol, and all of the chick embryo and body fluid were removed. The resulting egg shell is cut into about 8 mm long and 8 mm wide while maintaining villi, allantois adhered to the inner surface of egg shell, and put them in a 24-well culture plate piece by piece. The culture medium was prepared by (i) mixing 199 medium (GIBCO-BRL, NY, USA) with F10 medium (GIBCO-BRL, NY, USA) at a ratio of 1:1, (ii) adding 0.075% of sodium bicarbonate and 100 μg/Ml of gentamicin.

To the 10˜11-day-old SPF embryonated egg (Sunrise Co., NY) was infected with virus by adding 100 μl of crude allantoic fluid KBNP-0028 prepared in Example 2-1, which is 4-10-fold diluted to the surface of villi, allantois of hatchery egg shell fragments, and culturing for 30 min at 37° C. After adding 1000 μl of the culture medium, Alnus japonica extracts was added to 6 well plates, respectively to the concentration of 400, 300, 200 and 100 μg/Ml The virus-infected fluid containing Alnus japonica extracts was cultured for 7 days at 37° C.

### 2-3: Test of Antiviral Effect

Culture broth of said virus-infected fluid containing Alnus japonica extracts at each concentration, prepared in Example 2-2 was taken to carry out plate hemagglutination test. 25 μl of the culture broth and 25 μl of chicken red blood cells (0.1%) were dropped on glass plate in the same amount and mixed evenly. The virus proliferation was examined according to whether hemagglutination was formed within 2 min after moving the glass plate right and left, and up and down. It is confirmed that in the case of treating with the leaves and stems (duramen) extract of Alnus japonica, virus proliferation was completely inhibited without toxicity to cell in a concentration of 400 μg/Ml and partial antiviral effect was shown in 300 μg/Ml. In the case of treating with stems (bark) extract of Alnus japonica, virus proliferation was completely inhibited without cytotoxicity in concentrations down to 300 μg/Ml, and partial antiviral effect was shown in 200 μg/Ml. In the case of treating with flowers extract of Alnus japonica, complete virus inhibition effect was shown in concentrations down to 200 μg/Ml, and partial virus inhibition effect was shown in 100 μg/Ml (Table 1).

TABLE 1
Antiviral effect of Alnus japonica extract

| Control | | | Concentration of Alnus | | | |
|---|---|---|---|---|---|---|
| | No- | Site (number of | japonica extract (μg/Ml) | | | |
| Virus | virus | hemagglutination/sample) | 400 | 300 | 200 | 100 |
| 6/6 | 0/6 | Leaves (number of | 0/6 | 0/6 | 2/6 | 6/6 |
| | | hemagglutination/sample) | | | | |
| | | Stems (number of | 0/3 | 0/3 | 0/3 | 0/3 |
| | | hemagglutination/sample) | | | | |
| | | Flowers (number of | 0/3 | 0/3 | 0/3 | 0/3 |
| | | hemagglutination/sample) | | | | |

*NT = Not tested

### 2-4: MTT Assay

To confirm cytotoxicity of Alnus japonica extract, Alnus japonica extracts prepared in example 2-2 were put into 24 well plates at concentrations of 400, 300, 200 and 100 μg/Ml. Chorioallantoic membrane was attached to egg shell fragment of each well of the 24 well plate and then transferred to each well of a 96 well plate, followed by addition of 40 μl of MTT solution (MTT 0.5% aqueous solution), and incubated for 1˜3 hrs at 37° C. After 120 μl of DMSO was added and stirred for 30 min, then the result was read at 562 nm wavelength with ELISA (Table 2). As a result, in the case of treating with the leaves, stems (duramen) and stems (bark) extract of Alnus japonica, the measured value was similar to or higher than the MTT OD value of a control group added only with virus (0.381±0.057), thus confirming that Alnus japonica extracts have no cytotoxicity. However, in the case of treating with the flowers extract of Alnus japonica, the value being lower than the MTT OD value of the control group, it confirmed that the flowers extract of Alnus japonica showed cell toxicity. Therefore, it was concluded that stem (bark) was the most proper material as an antiviral agent.

TABLE 2
Cell toxicity of Alnus japonica extract

| | | Site (MTT OD | Concentration of Alnus japonica extract | | | |
|---|---|---|---|---|---|---|
| Control | | mean ± standard | (μg/Ml) | | | |
| Virus | No-virus | deviation) | 400 | 300 | 200 | 100 |
| 0.381 ± 0.057 | 0.403 ± 0.118 | Leaves (MTT OD | 0.406 ± 0.037 | 0.409 ± 0.050 | 0.942 ± 0.203 | 1.156 ± 0.023 |
| | | mean ± standard | | | | |
| | | deviation) | | | | |
| | | Stems (duramen) | 0.309 ± 0.045 | 0.392 ± 0.108 | 0.409 ± 0.005 | 0.390 ± 0.088 |
| | | (MTT OD mean ± | | | | |
| | | standard deviation) | | | | |
| | | Stems (bark) | 0.844 ± 0.113 | 0.690 ± 0.123 | 0.521 ± 0.074 | 0.570 ± 0.078 |
| | | (MTT OD mean ± | | | | |
| | | standard deviation) | | | | |
| | | Flowers (MTT OD | 0.288 ± 0.053 | 0.290 ± 0.029 | 0.239 ± 0.024 | 0.226 ± 0.047 |
| | | mean ± standard | | | | |
| | | deviation) | | | | |

### INDUSTRIAL APPLICABILITY

As described above in detail, Alnus japonica extracts according to the present invention have a low toxicity to choriollantonic cell which is a normal cell, while having an excellent antiviral effect. Therefore, the composition comprising Alnus japonica extracts can be used effectively in foods and pharmaceutical compositions since it is effective and safe in preventing and treating influenza virus diseases.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A method for treatment or prevention of influenza in a subject suffering or susceptible to same, said method comprising administering to said subject a food composition comprising Alnus japonica extracts.

2. The method of claim 1, wherein said subject is a human, pig, horse or bird.

3. A method for treatment or prevention of influenza in a subject suffering or susceptible to same, said method comprising administering to said subject a pharmaceutical composition comprising Alnus japonica extracts as an active ingredient.
